(19) United States
(12) Patent Application Publication
(10) Pub. No.: US 2018/0140526 A1
(43) Pub. Date: May 24, 2018

(54) COMPOSITION FOR HYDRATION IN SOLID FORM

(71) Applicant: Mayra V. Aruca, Century City, CA (US)

(72) Inventor: Mayra V. Aruca, Century City, CA (US)

(21) Appl. No.: 15/794,545

(22) Filed: Oct. 26, 2017

Related U.S. Application Data
(60) Provisional application No. 62/413,670, filed on Oct. 27, 2016.

(51) Int. Cl. A61K 8/60; A61K 8/19; A61K 8/11; A61Q 19/00

(52) U.S. Cl. CPC A61K 8/60; A61Q 19/007; A61K 8/11; A61K 8/19

(57) ABSTRACT

A composition for aiding the hydration of skin. The composition includes sodium, potassium and dextrose of water, and the composition is in solid form.

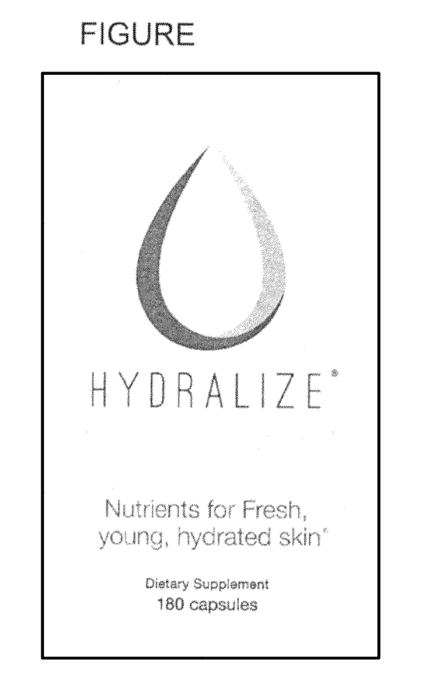

The present application claims priority from U.S. Provisional 62/413,670 filed on Oct. 27, 2016, the entire contents of which is incorporated herein by reference.

### BACKGROUND

The composition of the disclosure aids in hydrating skin and overall beauty. Due to today's fad diets, caffeine addiction through the popularity of coffee and its ensuing exhaustion of adrenal glands, and our fast paced lifestyle, people often not only feel tired and haggard, but are also looking it. There is a process to skin hydration. We are often erroneously told that hydration can be achieved by drinking lots of water but this is not the case. Hydration of the skin cannot be fully accomplished by only drinking water. The water is not absorbed by the body unless specific ingredients are present for the absorption to take place. If these ingredients are not present the water will be expelled from the body through urination and the symptoms of dehydration such as dry, loose, wrinkly and deflated skin will continue.

While various drinks have been promoted to aid in hydration, a composition in the form of a solid that can be administered orally has not been contemplated, further still nothing like it has previously been created in the beauty industry for the beautifying results ensuing from this hydration. Thus, the composition solves the problem of a lack of delivery of water and hydration to the skin's surface by supplying the ingredients necessary in the absorption of water to skin for hydration, which ultimately leads to a younger more esthetically pleasing look. For example, by hydrating the facial skin, the person's face will have a plumper, younger look. This is due to the body's ability to properly absorb water because it has received the ingredients necessary to do so through the injestion of the composition.

### SUMMARY OF THE INVENTION

The composition of the disclosure relates to the hydration of skin. The ingredients in the composition of the disclosure aid in water consumption, water retention/absorption, and reversing the effects of dehydration. It was discovered that a blend of the ingredients would aid the body in maintaining hydrated facial skin. Indeed, water does not hydrate our skin; rather it is how water is absorbed by our skin based on other ingredients in our system. Accordingly, the composition of the disclosure works with the renal system to help the body retain some of the water it loses and to hydrate skin from the inside out. For example, as reported in The Lancet, British Medical Journal (Aug. 5, 1987), it was discovered that sodium transport and glucose transport are coupled in the small intestine so that glucose accelerates absorption of solute and water. In addition, some observable effects of the composition of the disclosure are hydration, toning, lifting, or firming. For example, the International Journal of Cosmetic Science (2007) reported that drinking 2.25 liters of water every day for four weeks altered skin density and thickness. Thus, the composition in the form of a solid helps in water absorption along with the consumption of water for skin density improvement. Additionally, due to the effects of the composition, the use of lotions or creams might not be needed.

In addition, when, for example, the composition is taken with 8 fluid ounces of water it has the same effect as consuming a hydrating drink such as Pedialyte® or an anti-hangover drink. This concept has not been applied in the field of beauty. Until the composition of the present disclosure, there has not been a composition in the form of a solid that aids in hydration and furthermore carries its weight into the beauty industry based on the effects this hydration has on facial skin.

In one aspect, the composition is in a solid form that can be administered orally. In another aspect, the composition includes sodium, potassium, and dextrose. In another aspect, there is a method of producing the composition in the solid form.

### BRIEF DESCRIPTION OF THE DRAWINGS

The FIG. is a sample packaging label.

### DETAILED DESCRIPTION OF THE INVENTION

The composition of the disclosure is in the form of solid that can be administered orally. For example, the composition of the disclosure is in the form of a tablet or capsule.

The composition of the disclosure includes ingredients such as sodium, potassium and dextrose important in the absorption of water. That is, the ingredients in the composition aid in water consumption, water absorption water retention, and diminishing the effects of dehydration. For example, the composition mainly includes magnesium, sodium, potassium citrate, dextrose, chloride, citrate, and zinc. Other ingredients, such as vegetable cellulose, vegetable stearate and rice flour can be included. The composition is vegetarian and free of citrus, corn, gluten and yeast. For example, a preferred embodiment of the composition includes 120-160 mg magnesium, 625-695 mg sodium, 380-420 mg potassium citrate, 20-40 g dextrose, 130-170 mg chloride, 1-10 Meq citrate, and 0.1-10 mg zinc. A more preferred embodiment of the composition includes 140 mg magnesium, 675 mg sodium, 400 mg potassium citrate, 10 g dextrose, 150 mg chloride, 5 Meq citrate, and 2 mg zinc. The packaging may include the following example of a supplement facts.

Supplement Facts
Serving size: 2 Capsules
Servings Per Container: 90

Amount Per Serving
Magnesium
(as Magnesium Citrate) 140 mg.
Sodium: 675 mg.
Potassium Citrate: 400 mg.
Dextrose (glucose): 10 g
Chloride: 150 mg.
Citrate: 5 Meq
Zinc: 2 mg.
Other ingredients: Vegetable cellulose, vegetable
stearate, rice flour.
This product is vegetarian and free of
citrus, corn, gluten and yeast.
Warnings: Talk to your doctor before using hydralize if
you have heart or kidney disease, electrolyte or fluid restrictions.
If you are pregnant or nursing, consult your
health care practitioner before taking this product.
Keep out of reach of children. Store container tightly
closed in a cool, dry place.

The composition can be encapsulated in capsules. The empty capsules can have a volume capacity of from 0.1 ml to 1.4 ml, preferably 0.13 ml to 1.37 ml. In addition, the empty capsules can have a closed length of from 10 mm to 27 mm. Further, the empty capsule can have a diameter of 4.5 to 10 mm, preferably 4.9 to 9.9 mm. For example, commercially available capsules such as capsules sold by Capsugel® can be used.

The composition may be administered two or three times daily. Administration of three capsules three times a day is preferred. Administration of three capsules three times a day with eight ounces of water each time is preferred. Preferably, the composition is not mixed with alcohol or caffeinated products to avoid diminishing the effects of the composition. The composition can be used as a dietary supplement for adults. The packaging may include the following example of a suggested use.

Suggested Use

As a dietery supplement for adults, take 3 capsules
three times a day with eight ounces of water each time.
Caution: High doses of magnesium can have a
laxative effect.
Magnesium is an essential mineral involved in over
300 metabolic reactions. It also supports muscle
relaxation.
↑
↑ This statement has not been evaluated by the Food and Drug Administration. This product is not intended to diagnose, treat, cure or prevent disease.

The process of preparing the composition in solid form is encompassed. For example, if the composition is in a capsule form, it can be manufactured by the following process: the raw materials are weighed and blended to obtain a composition; the composition is encapsulated, hitting the preferred weight(s), to obtain capsules; and the capsules are packaged. The process is performed at room temperature. Preferred specific amounts of the ingredients may be used in unit amounts. For example, each ingredient is carefully inspected and placed into a blender one by one, then the mixture is blended and ready to be encapsulated. The ingredients can be blended as follows:

| | | |
|---|---|---|
| Sodium Ascorbate: | 225.0000 | mg |
| Potassium Citrate: | 133.3333 | mg |
| 30% Dextrose: | 3.3333 | mg |
| Chloride (Sodium Chloride): | 50.0000 | mg |
| Citric Acid: | 1.6666 | mg |
| Magnesium Chelate 15%: | 13.3333 | mg |
| Zinc Chelate 10%: | 0.6666 | mg |
| Magnesium Stearate: | 10.0000 | mg |
| Rice Flour: | 2.6669 | mg |

The composition is then encapsulated, hitting the preferred weight(s), to obtain capsules, and the capsules are packaged. For blending, a Kelly Paterson V-Blender, such as a 30 cubic foot that can blend 800 kg or a 10 cubic foot that can blend 200 kg can be used. For encapsulation, empty shell capsules can be used. The packaging can be done by conveying the capsules or other solid form into bottles. Tablet forms can be prepared in a similar manner.

While the composition of the disclosure has been depicted and described with reference to exemplary aspects, such reference does not imply a limitation on the invention, and no such limitation is to be inferred. The composition of the disclosure is capable of considerable modification, alteration, and equivalents in form and function, as will be apparent to those of ordinary skill in the pertinent art having the benefit of this disclosure. Consequently, the composition of the disclosure is intended to be limited only by the spirit and scope of the appended claims, giving full cognizance to equivalence in all respects. All references cited herein are hereby incorporated by reference in their entirety.

What is claimed is:

1. A composition comprising sodium, potassium and dextrose.

2. A method of manufacturing comprising preparing a composition comprising sodium, potassium and dextrose, and encapsulating the composition.

3. A capsule manufactured by the method of claim 2.

4. A capsule comprising a composition comprising sodium, potassium and dextrose.
